(19) United States
(12) Patent Application Publication
(10) Pub. No.: US 2022/0084100 A1
(43) Pub. Date: Mar. 17, 2022

(54) DIET PERFORMANCE TRACKING AND CONTENT

(71) Applicant: AmVentureX, Inc., Minneapolis, MN (US)

(72) Inventors: Matthew Payne, Edina, MN (US); Vincent Logan Delgado, San Marcos, TX (US); Jason Bright, Minneapolis, MN (US)

(21) Appl. No.: 17/471,519

(22) Filed: Sep. 10, 2021

Related U.S. Application Data
(60) Provisional application No. 63/077,005, filed on Sep. 11, 2020.

(51) Int. Cl. G06Q 30/06; G16H 20/60; G16H 20/30; G06F 16/9535; G06F 16/955; G09B 19/00

(52) U.S. Cl. CPC G06Q 30/0631; G16H 20/60; G16H 20/30; G09B 19/0092; G06F 16/9535; G06F 16/9558; G06Q 30/0633

(57) ABSTRACT

A computer-implemented method for diet performance tracking includes receiving one or more diet performance parameters from a first user and displaying these one or more diet performance parameters at a first user device. It also includes displaying, at the first user device, one or more diet challenges and receiving a selection from the first user, at the first user device, of a diet challenge. After receiving the selection of the diet challenge, the method includes displaying, at the first user device, video and audio content corresponding to the selected diet challenge. Also after receiving the selection of the diet challenge, the method includes displaying, at the first user device, one or more diet performance parameters received from the first user and displaying, at the first user device, one or more diet parameters corresponding to a second user at a location remote from the first user.

### RELATED APPLICATION

This application claims priority to U.S. provisional patent application No. 63/077,005 filed on Sep. 11, 2020.

### TECHNICAL FIELD

This disclosure generally relates to devices, systems, and methods for dieting performance tracking, diet coaching, and related dieting content. Embodiments disclosed herein can provide devices, systems, and methods that allow for tracking and displaying one or more diet performance parameters associated with a user and providing content, via a network, related to the one or more tracked diet performance parameters.

### BACKGROUND

Diet programs tend to be based on a one-size-fits-all approach. In general, these diet programs guess when a user's body enters a relevant diet state, such as ketosis and deep ketosis, by applying a common time factor to each user. However, because anatomical/physiological functions can vary significantly from person-to-person, these diet programs can be inaccurate and inefficient.

Other diet programs require various, complex inputs, such as calorie or macronutrient targets, and outputs are limited to weight and/or waist size. These diet programs present challenges for a user because a user must lookup caloric data on everything he or she consumes and, if caloric information is unavailable, the user must input a “best guess.” Moreover, outputs such as weight and/or waist size tend to be undesirable information for a user to share with others and, thus, for these diet programs, it can be difficult to discern whether that user is properly following the diet program. Consequently, users often give up on the diet program without seeing progress.

### SUMMARY

In general, various embodiments relating to devices, systems, and methods for dieting performance tracking, diet coaching, and related dieting content are disclosed herein. In particular, disclosed herein are embodiments utilizing diet performance parameters pertaining to an individual user. Such diet performance parameters can include, for example, one or more of glucose level, energy level, ketone level, fasting time, heart rate, respiration rate, and/or heart rate variability. These diet performance parameters can be displayed for a user to track diet performance in a manner specific to that individual user. These diet performance parameters can also be used for a diet challenge, selected by the user and received over a network, and compared to corresponding diet performance parameters of another user and/or diet performance benchmark(s) set by an instructor of the diet challenge.

Research has shown that social connectedness can result in greater weight loss and health outcomes for users. Embodiments disclosed herein can provide a platform that combines social connectedness, via a network, with dieting using diet performance parameters pertaining to an individual user. As such, embodiments disclosed herein can utilize networked connectedness combined with diet performance parameters (e.g., glucose level, energy level, ketone level, fasting time, heart rate, respiration rate, and/or heart rate variability) measured specific to an individual user.

Embodiments disclosed herein can take a novel approach to dieting by utilizing a user's biomarkers (e.g., quantitative or qualitative characteristics of biological processes) to show a user's progress and fine tune the user's diet. As one such example, a user who eats a carbohydrate rich meal can measure his or her blood glucose level to see how that food spikes his or her blood sugar level. This gives the user instant feedback, based on one or more of that user's body specific biomarkers, to know what he or she should avoid in the future. As another such example, a user who is intermittent fasting can measure his or her ketone levels to see when his or her body enters a fat burning state. As a further example, a user who is trying to eat in a caloric deficit, can track his or her heart rate during exercise to see how rigorous it was and how many calories were burned. A combination of these biomarkers, specific to that user's body, can be combined to determine what is happening in the body, often times before any progress with weight or waist size can be seen. This, in turn, can facilitate user motivation to stick with the diet to the point where, for instance, weight or waist size progress is evident.

In addition to this novel approach to dieting, embodiments disclosed herein can provide a community component, over a network, to further spur greater health outcomes for users. For example, in certain embodiments disclosed herein, a user can join a community of one or more other users, over a network, and within which the user can share his or her progress, via the network, relative to that user's individual measured biomarkers, rather than via that user's weight and/or waist size. As a further example, the community can include instructor content providing motivation and informational data to users, over a network, who have joined that community. This can provide users in the community the advantage of hearing from an expert on ways to tune their diet to hit certain biomarkers.

In some embodiments disclosed herein, certain biomarkers can also be gamified, further adding to the experience of the user. The gamification can be provided, over a network, upon a user gamification request. As one example, a user who is attempting an extended fast can join a community of one or more other users and share, via the network, that user's fasting progress with those one or more other users in the community. In addition, the community can provide feedback, via the network, to that user as to ways to manage one or more biomarkers (e.g., energy).

One embodiment includes a computer-implemented method for diet performance tracking. This method embodiment includes the step of receiving one or more diet performance parameters from a first user and displaying these one or more diet performance parameters at a first user device. The one or more diet performance parameters can be, for example, one or more of glucose level, energy level, ketone level, fasting time, heart rate, respiration rate, and/or heart rate variability. The method can also include the step of displaying, at the first user device, one or more diet challenges, received over a network, and receiving a selection from the first user, at the first user device, of a diet challenge. After receiving the selection from the first user of the diet challenge, the method can further include displaying, at the first user device, video and audio content corresponding to the selected diet challenge received over the network. Also after receiving the selection from the first user of the diet challenge, the method can additionally include displaying, at the first user device, one or more diet performance parameters received from the first user and displaying, at the first user device, one or more diet parameters corresponding to a second user at a location remote from the first user. At least one of the diet parameters received from the first user and at least one of the diet parameters corresponding to a second user can be a same type of biomarker to facilitate a relative comparison.

Another embodiment includes a non-transitory computer-readable storage article having computer-executable instructions stored thereon to cause at least one programmable processor to receive one or more diet performance parameters from a first user and display these one or more diet performance parameters at a first user device. The one or more diet performance parameters can be, for example, one or more of glucose level, energy level, ketone level, fasting time, heart rate, respiration rate, and/or heart rate variability. The computer-executable instructions can further cause the at least one programmable processor to display, at the first user device, one or more diet challenges and to receive a selection from the first user, at the first user device, of a diet challenge. After receiving the selection from the first user of the diet challenge, the computer-executable instructions can further cause the at least one programmable processor to display, at the first user device, video and audio content corresponding to the selected diet challenge. Also after receiving the selection from the first user of the diet challenge, the computer-executable instructions can further cause the at least one programmable processor to display, at the first user device, one or more diet performance parameters received from the first user and display, at the first user device, one or more diet parameters corresponding to a second user at a location remote from the first user. At least one of the diet parameters received from the first user and at least one of the diet parameters corresponding to a second user can be a same type of biomarker to facilitate a relative comparison.

In some such embodiments, including those above, after receiving the selection from the first user of the diet challenge, the first user device can output, over a network, a diet challenge selection signal corresponding to the selected diet challenge and, in response to outputting the diet challenge selection signal, receive, at the first user device, video and audio content related to the selected diet challenge (e.g., from a remote server in communication with the first user device via the network). In one such embodiment, the video and audio content can be streamed, at the first user device, over the network. In another such embodiment, the video and audio content can be downloaded from a remote server and saved into memory at the first user device. In either case, the video and audio content can provide information related to one or more diet performance parameters, such as one or more of glucose level, energy level, ketone level, fasting time, heart rate, respiration rate, and/or heart rate variability.

An additional embodiment includes a non-transitory computer-readable storage article having computer-executable instructions stored thereon to cause at least one programmable processor to: receive, from a user, a diet challenge selection; based on the diet challenge selection received, present via a graphical user interface at least one diet performance parameter icon associated with a timeline; in response to the presented at least one diet performance parameter icon, receive user diet performance parameter input; and based on the diet challenge selection received, present via the graphical user interface a food shopping icon associated with the timeline, wherein the food shopping icon is configured to provide a list of recommended food items to be purchased.

In a further embodiment of this article, at least three diet performance parameter icons, associated with the timeline, are presented via the graphical user interface based on the diet challenge selection received. For example, the at least three diet performance parameter icons can include a ketone level input icon, a mood input icon, and a blood data input icon. The ketone level input icon, the mood input icon, and the blood data input icon can be presented, associated with the timeline, based on the diet challenge selection received being a ketosis diet challenge. In one further example, the at least three diet performance parameter icons can further include a user exercise icon, and the user exercise icon can be configured to present the user with a prompt instructing the user to perform a specified exercise.

In a further embodiment of this article, the list of recommended food items to be purchased includes food items that are associated with the diet challenge selection received. The food shopping icon can be further configured to provide a webpage link to a grocery store or food delivery service from which the list of recommended food items can be purchased. For example, the webpage link can be configured to direct a user to an online shopping cart prefilled with the list of recommended food items. In some such embodiments, the list of recommended food items to be purchased can be created by creating a food item sub-group from a larger food item main group, where the food item sub-group is created from the larger food item main group by selecting those food items, from the main group to be included in the food item sub-group, that are associated with the diet challenge selection received. For instance, the diet challenge selection received can be a ketosis diet challenge, and the food item sub-group can be created from the larger food item main group by selecting those food items, from the main group to be included in the food item sub-group, that are associated with the ketosis diet challenge.

Another embodiment includes a computer-implemented method for diet performance tracking. This method can include receiving, from a user, a diet challenge selection. This method also can include, based on the diet challenge selection received, presenting via a graphical user interface at least one diet performance parameter icon associated with a timeline. This method can further include, in response to the presented at least one diet performance parameter icon, receiving user diet performance parameter input. And, this method can include, based on the diet challenge selection received, presenting via the graphical user interface a food shopping icon associated with the timeline, where the food shopping icon provides a list of recommended food items to be purchased.

In a further embodiment of this method, at least three diet performance parameter icons, associated with the timeline, are presented via the graphical user interface based on the diet challenge selection received. The at least three diet performance parameter icons can include a ketone level input icon, a mood input icon, and a blood data input icon. The ketone level input icon, the mood input icon, and the blood data input icon can be presented, associated with the timeline, based on the diet challenge selection received being a ketosis diet challenge. In a further example, the at least three diet performance parameter icons can additionally include a user exercise icon, and the user exercise icon can be configured to present the user with a prompt, via the graphical user interface, instructing the user to perform a specified exercise.

In a further embodiment of this method, the list of recommended food items to be purchased includes food items that are associated with the diet challenge selection received. For instance, the food shopping icon can further provide a webpage link to a grocery store or food delivery service from which the list of recommended food items can be purchased. The webpage link can direct the user to an online shopping cart prefilled with the list of recommended food items. In some examples, the list of recommended food items to be purchased can be created by creating a food item sub-group from a larger food item main group, where the food item sub-group is created from the larger food item main group by selecting those food items, from the main group to be included in the food item sub-group, that are associated with the diet challenge selection received. In one such example, the diet challenge selection received is a ketosis diet challenge, and the food item sub-group is created from the larger food item main group by selecting those food items, from the main group to be included in the food item sub-group, that are associated with the ketosis diet challenge.

The details of one or more examples are set forth in the accompanying drawings and the description below. Other features, objects, and advantages will be apparent from the description and drawings.

### BRIEF DESCRIPTION OF THE DRAWINGS

The following drawings are illustrative of particular embodiments of the present invention and, therefore, do not limit the scope of the invention. The drawings are intended for use in conjunction with the explanations in the following description. Embodiments of the invention will hereinafter be described in conjunction with the appended drawings, wherein like numerals denote like elements. The features illustrated in the drawings are not necessarily to scale, though embodiments within the scope of the present invention can include one or more of the illustrated features (e.g., each of the illustrated features) at the scale shown.

FIG. 1 is an exemplary embodiment of a graphical user interface for diet performance tracking, coaching, and content display.

FIGS. 2A-2D illustrate an exemplary embodiment of a graphical user interface diet challenge sequence corresponding to a selected diet challenge. FIG. 2A shows a first graphical user interface of the diet challenge sequence corresponding to the selected diet challenge, FIG. 2B shows a second graphical user interface of the diet challenge sequence corresponding to the selected diet challenge, FIG. 2C shows a third graphical user interface of the diet challenge sequence corresponding to the selected diet challenge, and FIG. 2D shows a fourth graphical user interface of the diet challenge sequence corresponding to the selected diet challenge.

FIG. 3 a schematic diagram of an exemplary embodiment of a network, and exemplary devices in the network, for implementing various embodiments disclosed herein.

FIG. 4 is a flow diagram of an exemplary embodiment of a computer-implemented method for providing a graphical user interface diet challenge sequence, such as the exemplary embodiment illustrated at FIGS. 2A-2D.

### DETAILED DESCRIPTION

The following detailed description is exemplary in nature and is not intended to limit the scope, applicability, or configuration of the invention in any way. Rather, the following description provides some practical illustrations for implementing embodiments of the present invention. Examples of constructions, materials, and/or dimensions are provided for selected elements. Those skilled in the art will recognize that many of the noted examples have a variety of suitable alternatives.

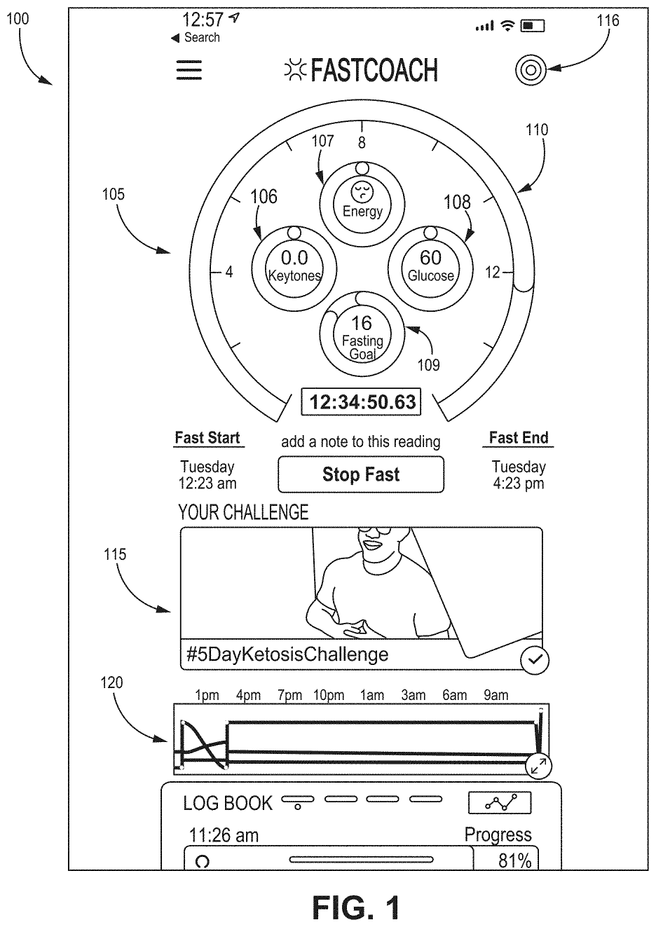

FIG. 1 shows an exemplary embodiment of a graphical user interface 100 for diet performance tracking, coaching, and content display.

The graphical user interface 100 can be displayed at a user device. For example, the user device can be a mobile computing device, such as a phone or tablet having at least one programmable processor and at least one non-transitory computer-readable storage. The user device can execute computer-executable instructions, such as an application, stored thereat to display the graphical user interface 100. More specifically, the user device can a have non-transitory computer-readable storage article having computer-executable instructions, such as the application, stored thereon to cause the at least one programmable processor of the user device to display the graphical user interface 100 at a display of the user device. The user device can also have the non-transitory computer-readable storage article having the computer-executable instructions, such as the application, stored thereon to cause the at least one programmable processor of the user device to receive a selection from the user and transmit the selection over a network (e.g., WiFi, cellular, etc.). For example, to facilitate wireless data exchange over the network, the user device can include a transceiver (e.g., a radio frequency transceiver).

The graphical user interface 100 can include one or more regions at which different types of information can be displayed to the user and/or different types of diet content, including different types of diet challenges, can be selected by the user.

In the illustrated embodiment, the graphical user interface 100 includes a diet performance parameter region 105. The diet performance parameter region 105 can display one or more diet performance parameters specific to the particular user of the user interface 100. The user device can receive one or more inputs related to one or more (e.g., each) of the diet performance parameters displayed at the graphical user interface 100, and the computer-executable instructions, such as the application, stored at the user device can cause at least one programmable processor of the user device to display the one or more diet performance parameters at the graphical user interface 100. The one or more diet performance parameters displayed at the diet performance parameter region 105 of the graphical user interface 100 can include one or more of the user's glucose level, energy level, ketone level, fasting time, heart rate, respiration rate, heart rate variability, oxygen levels, resting heart rate, steps, and activity level.

In some embodiments, the specific type(s) of diet performance parameters displayed at the diet performance parameter region 105 can correspond to those diet performance parameters used for a specific diet challenge previously selected by the user. For example, if a user selects a first diet challenge (e.g., a ketosis diet challenge) (e.g., selected by the user from the diet challenge region 115), the graphical user interface 100 can be configured to display a first set of diet performance parameters at the diet performance parameter region 105 corresponding to the selected first diet challenge (e.g., corresponding to the selected ketosis diet challenge, such that, for instance, the first set of diet performance parameters can be one or more of user ketone level 106, user energy level 107, user glucose level 108, and user fasting time 109). Similarly, if a user selects a second diet challenge (e.g., a low carb diet challenge) (e.g., selected by the user from the diet challenge region 115), the graphical user interface 100 can be configured to display a second set of diet performance parameters at the diet performance parameter region 105 corresponding to the selected second diet challenge (e.g., corresponding to the selected low carb diet challenge, such that, for instance, the second set of diet performance parameters can be one or more of user glucose level 108, user fasting time 109, and user food consumption data), where the displayed second set of diet performance parameters, corresponding to the selected second diet challenge, is a different set of diet performance parameters than the first set of diet performance parameters, corresponding to the selected first diet challenge (e.g., the first set of diet performance parameters includes at least one diet performance parameter different than the diet performance parameters of the second set of diet performance parameters).

In the illustrated embodiment of the graphical user interface 100, the diet performance parameter region 105 displays user ketone level 106, user energy level 107, user glucose level 108, and user fasting time 109. Each of the user ketone level 106, user energy 107, user glucose level 108, and user fasting time 109 are specific, and correspond, to the particular user of the graphical user interface 100. As noted, the user device can receive one or more inputs related to each of the diet performance parameters 106-109 displayed at the diet performance region 105, and the computer-executable instructions, such as the application, stored at the user device can cause at least one programmable processor of the user device to display each of the received diet performance parameters 106-109. For example, the user ketone level 106 can be received at the user device from a blood ketone meter (e.g., in communication with the user device), and the user glucose level 108 can be received at the user device from a blood glucose meter or a continuous glucose meter (e.g., via a wireless connection between the user device and each of the blood ketone meter and the blood glucose meter). As another example, the user energy level 107 can be received via user input at the user device, such as user input in response to an energy level input prompt, provided via the graphical user interface 100, to the user at the user device. For instance, the computer-executable instructions, such as the application, stored at the user device can cause at least one programmable processor of the user device to display an energy level input prompt at the user device (e.g., via the graphical user interface 100), such as including a plurality of different preset answers (e.g., a scale of one to five, with one being a lowest energy level and five being a highest energy level) to the energy level input prompt for the user to select. The user fasting 109 can also be received via user input at the user device, such as user input in response to a fasting input prompt to the user at the user device. For instance, the computer-executable instructions, such as the application, stored at the user device can cause at least one programmable processor of the user device to display a fasting start icon, at the graphical user interface 100, that can be selected by the user to start a fasting timer. In such an example, such as that shown here, the user fasting 109 can be displayed as an elapsed time, at the graphical user interface 100, since the user provided input to start the fasting timer and the elapsed fasting time can be input as a diet performance parameter 105.

In the illustrated embodiment, the diet performance region 105 also includes a fasting progress icon 110. The fasting progress icon 110 can provide a display of a current fasting status relative to a preset fasting end time. For example, the fasting progress icon 110 in the illustrated embodiment shows both the current elapsed fasting time as well as the proximity of the current elapsed fasting time relative to the preset fasting end time and, thus, also provides a visual representation of time remaining to the preset fasting end time. Here, the particular display is in the form of an arc, with one end of the arc representing the fasting start time and the opposite end of the arc representing the preset fasting end time such that the current elapsed fasting time is represented as a bar progressing from the fasting start time toward the preset fasting end time. As also shown here, the diet performance parameters 106-109 can be displayed within the boundary of the arc. Other diet performance parameters can be included in a similar manner, such as heart rate, respiration rate, heart rate variability, oxygen levels, resting heart rate, steps, and activity level.

In addition to the diet performance region 105, the graphical user interface 100 can include a diet challenge region 115 that can provide diet content received over a network, for instance in response to a user selection of a diet challenge. In one embodiment, the diet challenge region 115 can be displayed at a same screen of the graphical user interface 100 as the diet performance region 105 so that both the diet content at the diet challenge region 115 and the one or more diet performance parameters at the diet performance parameter region 105 can be seen at the same time. In another embodiment, the diet challenge region 115 can be displayed as a separate page of the graphical user interface 100 as the diet performance region 105. As one example, the graphical user interface 100 can include a challenge icon 116, and upon user selection of the challenge icon 116 the computer-executable instructions, such as the application, stored at the user device can cause at least one programmable processor of the user device to generate and display the diet challenge region 115 in response to user input selection of the challenge icon 116. For example, selection of the challenge icon 116 can cause the computer-executable instructions, such as the application, stored at the user device can cause at least one programmable processor of the user device to display (e.g., at the graphical user interface 100) one or more diet challenges for user selection. In such example, after receiving a selection of the challenge icon 116, the user can then select one specific diet challenge presented and, in response, the computer-executable instructions, such as the application, stored at the user device can cause at least one programmable processor of the user device to display the selected diet challenge at the diet challenge region 115.

The diet challenge region 115 can display, for example, live and/or archived diet content, for instance, received over a network at the user device from a remote server. The diet content displayed at the diet challenge region 115 can include audio and/or video content related to one or more of the diet performance parameters displayed at the diet performance region 105. For example, the diet content displayed at the diet challenge region 115 can include audio and/or video content related to one or more of glucose level, energy level, ketone level, fasting time, heart rate, respiration rate, heart rate variability, oxygen levels, resting heart rate, steps, and activity level displayed at the diet performance region 105. In some embodiments, the diet challenge region 115 can display video chat information received, at the user device, from a remote server. The video chat information can be received from an instructor of the diet challenge located at a remote location. And, the video chat information can provide instructor feedback related to the one or more of the diet performance parameters displayed at the diet performance region 105. In some such embodiment, the user can input user video chat information and send this user video chart information to the instructor via the remote server. As such, the diet challenge region 115 can be configured to facilitate real-time information exchange related to the one or more of the diet performance parameters displayed at the diet performance region 105, between a user and an instructor located at remote locations and, in this way, provide a coaching function for diet performance.

Thus, the graphical user interface 100 can display, at the first user device, one or more diet challenges, received over a network. A first user at the first user device can input a selection of a diet challenge and the first user device can receive the diet challenge selection from the first user. After receiving the diet challenge selection from the first user, the first user device can display, at the graphical user interface 100, video and audio content corresponding to the selected diet challenge received over the network. Also after receiving the selection from the first user of the diet challenge, the first user device can display, at the graphical user interface 100, one or more diet performance parameters relating to the first user (e.g., and corresponding to the selected diet challenge). At the same time, the first user device can also display, at the graphical user interface 100, one or more diet parameters corresponding to a second user at a location remote from the first user. At least one of the diet parameters relating to the first user and at least one of the diet parameters relating to the second user can be a same type of biomarker to facilitate a relative comparison between the first and second users (e.g., and the second user can have selected the same type of diet challenge selected by the first user). In one embodiment, the diet challenge received over the network and displayed at the first user device can provide content related to the comparison between at least one of the diet parameters relating to the first user and (e.g., the same) at least one of the diet parameters relating to the second user. For instance, the diet challenge can provide content at the first user device, via the graphical user interface 100, including one or more instructions for actions the first user can take to facilitate a change in one or more of the diet parameters relating to the first user.

Upon the first user device receiving a diet challenge selection from the first user, the first user device can output, over a network, a diet challenge selection signal corresponding to the selected diet challenge presented at the diet challenge region 115. And, in response to the first user device outputting the diet challenge selection signal, the first user device can receive thereat, over the network, video and audio content related to the selected diet challenge. In one such embodiment, the video and audio content can be streamed from a remote server to the first user device over the network. In another such embodiment, the video and audio content can be downloaded from the remote server to the first user device and saved into memory at the first user device. In either case, the video and audio content can provide information related to one or more diet performance parameters, such as one or more of glucose level, energy level, ketone level, fasting time, heart rate, respiration rate, and/or heart rate variability displayed at the diet performance parameter region 105.

The remote server can receive diet performance parameters and/or diet challenge selection signals from the first user device and send diet challenge content to the first user device over the network. The remote server can include a non-transitory computer-readable storage article having computer-executable instructions stored thereon to cause the at least one programmable processor of the remote server to send diet challenge content to the first user device, over the network, in response to receiving a diet challenge selection signal from the first user device. As one example, the remote server can include a diet challenge database module storing a plurality of diet challenge content corresponding to a plurality of diet challenges. Upon receiving the diet challenge selection signal from the first user device, the remote sever can search the diet challenge database module for diet challenge content corresponding to the diet challenge selection signal for the specific type of diet challenged selected. Once the remote server locates the corresponding diet challenge content, the remote server can send the diet challenge content to the first user device over the network. As another example, the remote server can include a relay module storing a directory database. The directory database can include data relating to diet challenge instructor devices (e.g., contact information for die challenge instructors) and corresponding diet challenge content. Upon receiving the diet challenge selection signal from the first user device, the remote sever can search the directory database to locate a diet challenge instructor device corresponding to the diet challenge selection signal from the first user device. Then, the relay module at the remote server can receive streaming diet challenge content from the located instructor device and send the streaming diet challenge content to the first user device from which the corresponding diet challenge selection signal was received. For example, as described further herein, the relay module at the remote server can receive a food ordering list from the located instructor device and send the food ordering list to the first user device from which the corresponding diet challenge selection signal was received.

In some embodiments, the graphical user interface 100 can include one or more diet performance tracking displays 120. The one or more diet performance tracking displays 120 can provide a visualization of one or more diet performance parameters over time. For example, in the illustrated embodiment, the diet performance tracking display 120 is a graphical display plotting each of user ketone level 106, user energy level 107, user glucose level 108, and user fasting 109 (e.g., on the y-axis) versus time (e.g., on the x-axis). Thus, while the diet performance parameter region 105 can provide a display of each of user ketone level 106, user energy level 107, user glucose level 108, and user fasting 109 at a specific point in time, the performance tracking display 120 can provide a display of each of user ketone level 106, user energy level 107, user glucose level 108, and user fasting 109 over a period of time.

In some embodiments, a selected diet challenge can incorporate both instantaneous one or more diet performance parameters (e.g., displayed at the diet performance parameter region 105) as well as tracked one or more diet performance parameters over time (e.g., displayed at the diet performance tracking display 120). For example, a selected diet challenge can provide content generated in response to both instantaneous diet performance parameters and tracked diet performance parameters. For instance, a diet challenge may provide content directed to changing one or more diet performance parameters to hit a preset diet performance threshold level that is determined based on the corresponding, respective one or more tracked diet performance parameters over time. In this way, such diet challenge content can be tailored relative to the first user's unique diet performance parameter levels and, thereby, be more likely to facilitate the first user achieving noticeable diet results.

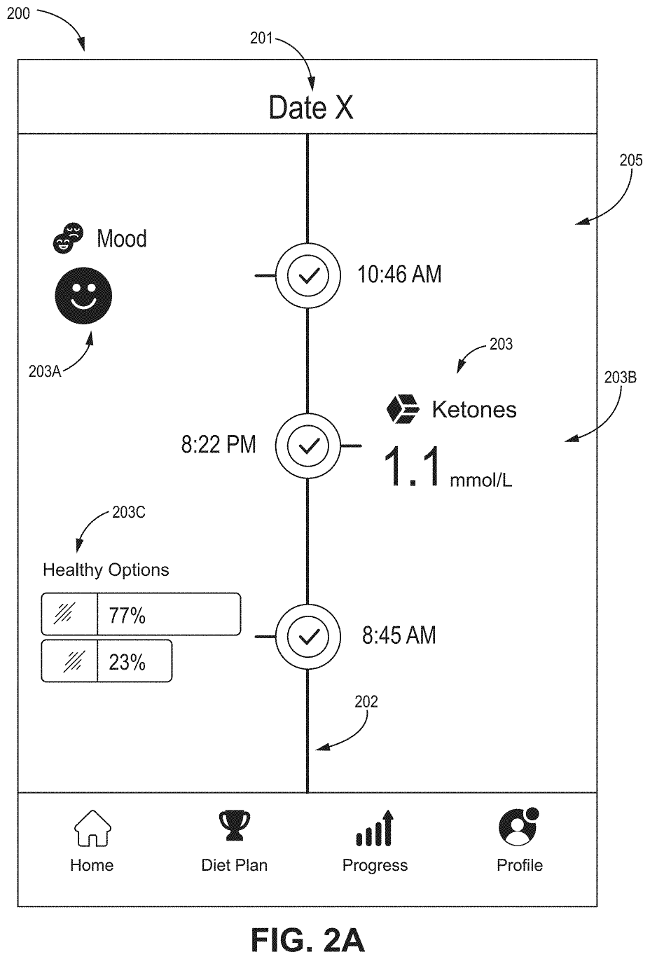

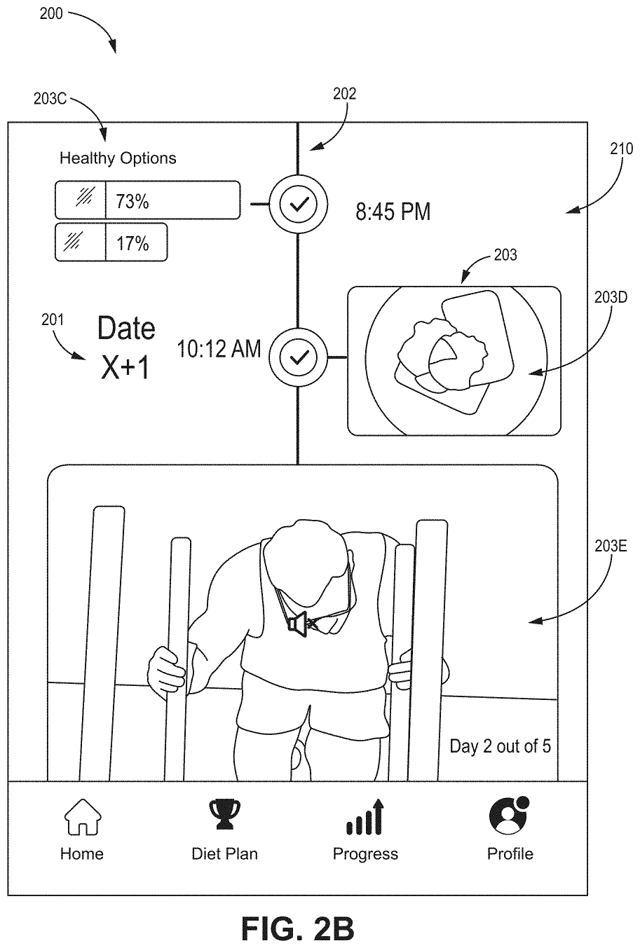

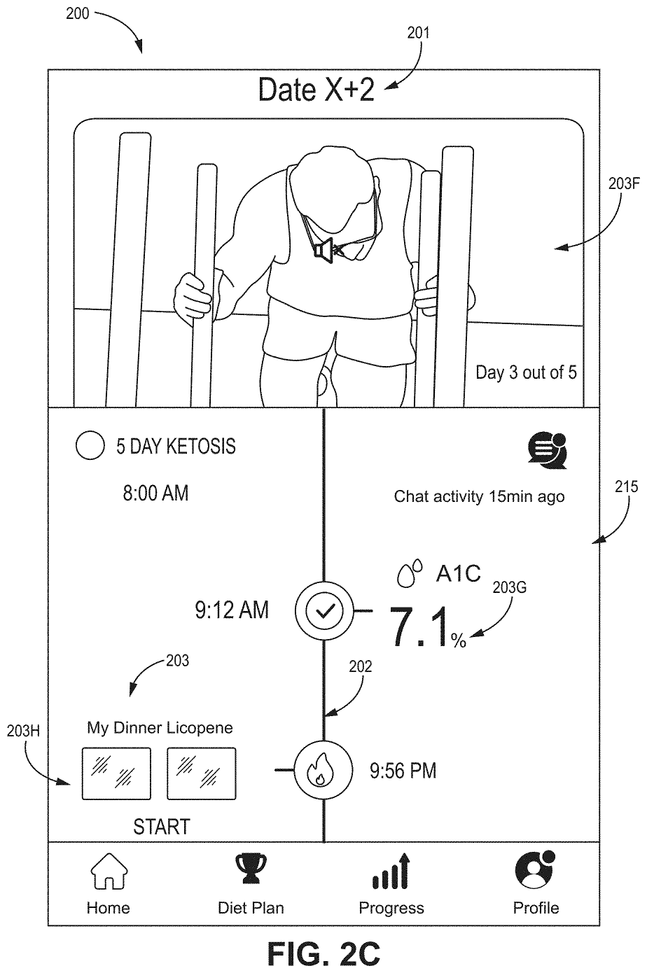

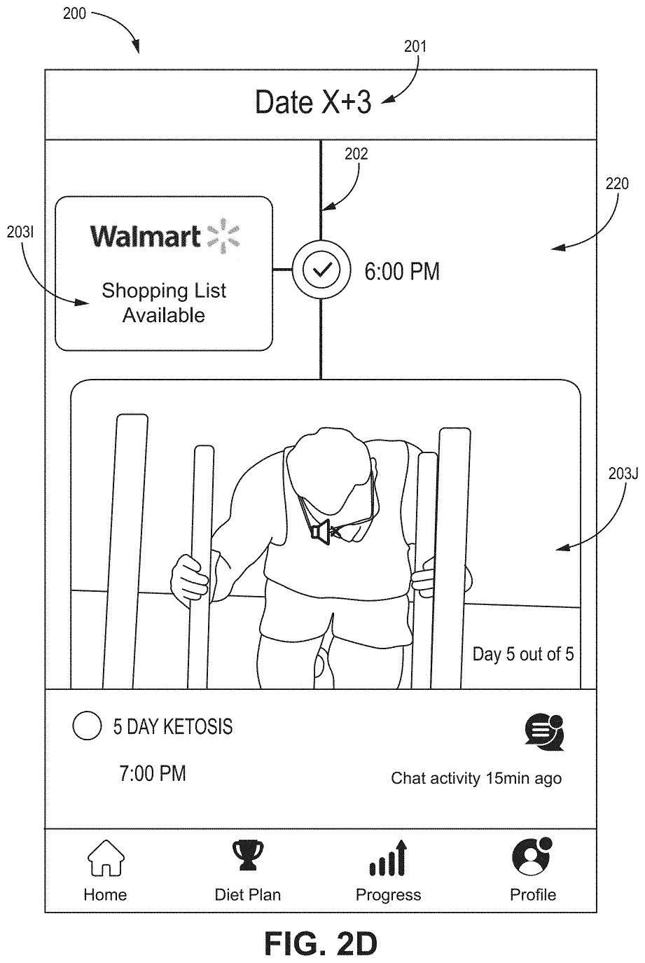

FIGS. 2A-2D illustrate an exemplary embodiment of a graphical user interface diet challenge sequence 200 corresponding to a selected diet challenge. FIG. 2A shows a first graphical user interface 205 of the diet challenge sequence 200 corresponding to the selected diet challenge, FIG. 2B shows a second graphical user interface 210 of the diet challenge sequence 200 corresponding to the selected diet challenge, FIG. 2C shows a third graphical user interface 215 of the diet challenge sequence 200 corresponding to the selected diet challenge, and FIG. 2D shows a fourth graphical user interface 220 of the diet challenge sequence 200 corresponding to the selected diet challenge.

As described previously, a user can select a specific diet challenge from a plurality of available diet challenges at the graphical user interface (e.g., selected at the graphical user interface by the user from the diet challenge region), and, in response to receiving the user selection of a specific diet challenge, the graphical user interface can be configured to display a diet challenge sequence corresponding to the specific diet challenge selected by the user at the graphical user interface. For example, the user interface can be configured to present the diet challenge sequence 200 embodiment shown at FIG. 2 in response to receiving user selection of a ketosis diet challenge. As such, the presented diet challenge sequence 200 can be a ketosis diet challenge sequence corresponding to the selected ketosis diet challenge. The user interface can be configured to present different types of diet challenge sequences corresponding to the different types of diet challenges available for user selection at the graphical user interface.

FIG. 2A shows the first graphical user interface 205 of the diet challenge sequence 200 corresponding to the selected diet challenge. As noted, the illustrated embodiment of the diet challenge sequence 200 is a ketosis diet challenge presented at the graphical user interface as a result of user selection of the ketosis diet challenge as the selected diet challenge. The first graphical user interface 205 can correspond to a first time period 201 in the diet challenge sequence 200. For example, here the first graphical user interface 205 corresponds to “Date X” which can be the first time period 201 in the ketosis diet challenge. For instance, the first time period 201 in the ketosis diet challenge can be a first day of the ketosis diet challenge.

The diet challenge sequence 200 can include a timeline 202 and one or more diet challenge sequence icons 203. In many embodiments, each of the one or more diet challenge sequence icons 203 can be associated with the timeline 202. For example, in the illustrated embodiment, the diet challenge sequence icons 203 include a first diet challenge sequence icon 203A, a second diet challenge sequence icon 203B, and a third diet challenge sequence icon 203C, and each of the first, second, and third diet challenge sequence icons 203A, 203B, 203C is associated with the timeline 202. In particular, in this embodiment, each of the first, second, and third diet challenge sequence icons 203A, 203B, 203C includes an indication as to a particular time on the timeline 202 where each of the first, second, and third diet challenge sequence icons 203A, 203B, 203C is located. Namely, the first diet challenge sequence icon 203A is located at a first time (e.g., “10:46 AM” of “Date X”) on the timeline 202, the second diet challenge sequence icon 203B is located at a second, later time (e.g., “8:22 PM” of “Date X”) on the timeline 202, and the third diet challenge sequence icon 203B is located at a third, later time (e.g., “8:45 PM” of “Date X”) on the timeline 202.

Each of the one or more diet challenge sequence icons 203 can correspond to a diet challenge sequence data point for the diet challenge sequence 200. For example, in the illustrated embodiment of the ketosis diet challenge, each of the first, second, and third diet challenge sequence icons 203A, 203B, 203C can correspond to a ketosis diet challenge sequence data point for the ketosis diet challenge selected by the user.

For instance, the first diet challenge sequence icon 203A can be a mood input icon. The mood input icon is associated with a first time of the timeline 202 and is configured to receive user input as to the user's mood (e.g., current mood). As one example, the mood icon can present the user, at the diet challenge sequence 200, with a list of a plurality of preset moods and the user can select (e.g., at the mood icon) one of the plurality of preset moods as the user's input for current mood. The user's input for current mood (e.g., the selected one of the plurality of preset moods presented by the mood icon) can then be transmitted via the network to the remote server and stored at the remote server for use in diet performance tracking analysis. Additionally, in some examples, the user's input for current mood can be displayed at the first diet challenge sequence icon 203A. For instance, as shown in FIG. 2A, the user's input for current mood can be a selection of a “happy” preset mood amongst other, different preset moods presented by the first diet challenge sequence icon 203A. The happy mood input from the user at the diet challenge sequence 200 can then be transmitted via the network to the remote server and, in some examples, the first diet challenge sequence icon 203A can then display a happy mood at the first diet challenge sequence icon 203A on the timeline 202.

The second diet challenge sequence icon 203B can be a ketone level input icon. The ketone level input icon is associated with a second, different time of the timeline 202 and is configured to receive user input as to the user's ketone level (e.g., current ketone level). As one example, the ketone level input icon can present the user, at the diet challenge sequence 200, with a ketone level measurement request. As one specific example, the ketone level input icon can present the user with a prompt instructing the user to measure the user's ketone level, and the user can then input, to the user device, the user's measured ketone level (e.g., measured via a ketone meter and input via wired or wireless communication between the ketone meter and the user device or input via user input). In response to receiving user input of the user's ketone level as a result of the prompt, the ketone level input icon can then display the user's measured ketone level (e.g., “1.1 mmol/L”). The user's input for ketone level can then be transmitted via the network to the remote server and stored at the remote server for use in diet performance tracking analysis. Additionally, in some examples, the user's input for ketone level can be displayed at the second diet challenge sequence icon 203B. Thus, ketone level input from the user at the diet challenge sequence 200 can be transmitted via the network to the remote server and, in some examples, the second diet challenge sequence icon 203B can then display the input ketone level at the second diet challenge sequence icon 203B on the timeline 202 (e.g., at a second time on the timeline 202 different than the first time on the timeline 202 where the first diet challenge sequence icon 203A is located).

The third diet challenge sequence icon 203C can be a food consumption icon. The food consumption icon is associated with a third, different time of the timeline 202 and is configured to receive user input as to the user's food consumption, for instance, at that third time on the timeline 202. As one example, the food consumption icon can present the user, at the diet challenge sequence 200, with a food consumption input request. As one specific example, the food consumption icon can present the user with a prompt instructing the user to input data relating to food the user has consumed at the associated third time on the timeline 202. In response to the prompt, the user can then input, to the user device, the data relating to the user's food consumption at the third time. The data relating to the user's food consumption can include calorie data associated with food consumed by the user and/or specific type(s) of food consumed by the user. For instance, the food consumption icon can present the user, at the diet challenge sequence 200, with a list of a plurality of preset types of food and the user can select (e.g., at the food consumption icon) one of the plurality of preset types of food as the user's input for current food consumption. In some such examples, the types of food included on the list of the plurality of preset types of food can be those food types corresponding to particular diet challenge previously selected by the user. In response to receiving user input of the user's food consumption as a result of the prompt, the food consumption icon can then display data relating to the input food consumption. For example, as shown for the food consumption icon 203C in FIG. 2A, the food consumption icon can display one or more metrics derived from the user's input food consumption, such as a proportion of the food consumed by the user that is classified as “healthy” (e.g., 77%) and a proportion of the food consumed by the user that is classified as “non-healthy” (e.g., 23%). In some cases, food consumed by the user can be classified as “healthy” or “non-healthy” based on the specific type of diet challenge previously selected by the user. The user's input for food consumption can then be transmitted via the network to the remote server and stored at the remote server for use in diet performance tracking analysis. Thus, food consumption input from the user at the diet challenge sequence 200 can be transmitted via the network to the remote server and, in some examples, the third diet challenge sequence icon 203C can then display data relating to the input food consumption at the third diet challenge sequence icon 203C on the timeline 202 (e.g., at a third time on the timeline 202 different than the first and second times on the timeline 202 where the first and second diet challenge sequence icons 203A, B are respectively located).

FIG. 2B shows the second graphical user interface 210 of the diet challenge sequence 200 corresponding to the selected diet challenge—a ketosis diet challenge as the selected diet challenge in the illustrated embodiment. The second graphical user interface 210 can correspond to a second time period 201 in the diet challenge sequence 200. For example, here the second graphical user interface 210 corresponds to “Date X+1” which can be the second time period 201 in the ketosis diet challenge. For instance, the first time period in the ketosis diet challenge can be a first day of the ketosis diet challenge, and the second time period in the ketosis diet challenge can be a second day of the ketosis diet challenge. The timeline 202 can continue from the first graphical user interface 205 to the second graphical user interface 210 since the sequence shown at the second graphical user interface 210 can be a continuance of the sequence shown at the first second graphical user interface 205.

For example, in the illustrated embodiment, the diet challenge sequence icons 203 include a fourth diet challenge sequence icon 203D and a fifth diet challenge sequence icon 203E, and each of the fourth and fifth diet challenge sequence icons 203D, 203E is associated with the timeline 202. In particular, in this embodiment, each of the fourth and fifth diet challenge sequence icons 203D, 203E includes an indication as to a particular time on the timeline 202 where each of the fourth and fifth diet challenge sequence icons 203D, 203E is located. Namely, the fourth diet challenge sequence icon 203D is located at a fourth time (e.g., “10:12 AM” of “Date X+1”) on the timeline 202, the fifth diet challenge sequence icon 203E is located at a fifth, later time (e.g., “12:20 PM” of “Date X+1”) on the timeline 202.

As noted, each of the one or more diet challenge sequence icons 203 can correspond to a diet challenge sequence data point for the diet challenge sequence 200. For example, in the illustrated embodiment of the ketosis diet challenge, each of the fourth and fifth diet challenge sequence icons 203D, 203E can correspond to a ketosis diet challenge sequence data point for the ketosis diet challenge selected by the user.

For instance, the fourth diet challenge sequence icon 203D can be a food consumption icon. The food consumption icon is associated with a fourth, different time of the timeline 202 and is configured to receive user input as to the user's food consumption, for instance, at that fourth time on the timeline 202. The fourth diet challenge sequence icon 203D can thus be similar to, or the same as, that described previously for the third diet challenge sequence icon 203C, which can also be a food consumption icon.

The fifth diet challenge sequence icon 203E can be a user exercise icon. The user exercise icon is associated with a fifth, different time of the timeline 202 and is configured to provide user exercise instruction and/or receive user exercise data as to the user's exercise, for instance, at that fifth time on the timeline 202. As one example, the user exercise icon can present the user, at the diet challenge sequence 200, with an exercise input request. As one specific example, the user exercise icon can present the user with a prompt instructing the user to perform a specified exercise, and the user can then input, to the user device, confirmation that the user has performed the specified exercise. In some cases, the user exercise icon can prompt the user to input a user's heartrate, for instance, while performing the specified exercise (e.g., heartrate measured via a pulse meter and input via wired or wireless communication between the pulse meter and the user device or input via user input). In response to receiving user input of the user's exercise as a result of the prompt, the user exercise icon can then display data relating to the user's performed exercise (e.g., the user's measured heartrate). The user's input for exercise, at the user exercise icon on the timeline 202, can then be transmitted via the network to the remote server and stored at the remote server for use in diet performance tracking analysis. As another example, in some embodiments, such as that illustrated for the second graphical user interface 210 at FIG. 2B, the user exercise icon can provide user exercise instruction, such as workout video and/or audio content to coach/instruct a user through a workout, such as that described previously herein for the diet challenge region with respect to FIG. 1.

As noted previously, the user exercise icon, like each of the other diet challenge sequence icons, can present data that corresponds to the diet challenge selected by the user. So, for instance, when the user selects a ketosis diet challenge, for example, the user exercise icon, like each of the other diet challenge sequence icons, can provide data that can be different from that provided by the user exercise icon, and each of the other diet challenge sequence icons, when another, different diet challenge is selected by the user. In this way, the diet challenge sequence 200 can track user diet performance, and provide user diet coaching, using diet performance parameters that are measured specific to the user and tailored to the specific considerations for the particular diet challenge the user has selected. This, in turn, can lead to improved diet results and increased user participation in the diet challenge.

FIG. 2C shows the third graphical user interface 215 of the diet challenge sequence 200 corresponding to the selected diet challenge—a ketosis diet challenge as the selected diet challenge in the illustrated embodiment. The third graphical user interface 215 can correspond to a third time period 201 in the diet challenge sequence 200. For example, here the third graphical user interface 215 corresponds to “Date X+2” which can be the third time period 201 in the ketosis diet challenge. For instance, the first time period in the ketosis diet challenge can be a first day of the ketosis diet challenge, the second time period in the ketosis diet challenge can be a second day of the ketosis diet challenge, and the third time period in the ketosis diet challenge can be a third day of the ketosis diet challenge. The timeline 202 can continue from the first graphical user interface 205 to the second graphical user interface 210 to the third graphical user interface 215 since the sequence shown at the third graphical user interface 215 can be a continuance of the sequence shown at the first and second second graphical user interfaces 205, 210.

For example, in the illustrated embodiment, the diet challenge sequence icons 203 include a sixth diet challenge sequence icon 203F, seventh diet challenge sequence icon 203G, and an eight diet challenge sequence icon 203H, and each of the sixth, seventh, and eighth diet challenge sequence icons 203F, 203G, 203H is associated with the timeline 202. In particular, in this embodiment, each of the sixth, seventh, and eighth diet challenge sequence icons 203F, 203G, 203H includes an indication as to a particular time on the timeline 202 where each of the diet challenge sequence icons 203F, 203G, 203H, is located. Namely, the sixth diet challenge sequence icon 203F is located at a sixth time (e.g., “8:00 AM” of “Date X+2”) on the timeline 202, the seventh diet challenge sequence icon 203G is located at a seventh, later time (e.g., “9:12 AM” of “Date X+1”) on the timeline 202, and the eighth diet challenge sequence icon 203H is located at an eighth, later time (e.g., “9:56 PM” of “Date X+2”).

As noted, each of the one or more diet challenge sequence icons 203 can correspond to a diet challenge sequence data point for the diet challenge sequence 200. For example, in the illustrated embodiment of the ketosis diet challenge, each of the sixth, seventh, and eighth diet challenge sequence icons 203F, 203G, 203H can correspond to a ketosis diet challenge sequence data point for the ketosis diet challenge selected by the user.

For instance, the sixth diet challenge sequence icon 203F can be a user exercise icon. The user exercise icon is associated with a sixth, different time of the timeline 202 and is configured to provide user exercise instruction and/or receive user exercise data as to the user's exercise, for instance, at that sixth time on the timeline 202. The sixth diet challenge sequence icon 203F can thus be similar to, or the same as, that described previously for the fifth diet challenge sequence icon 203E, which can also be a user exercise icon.

The seventh diet challenge sequence icon 203G can be a blood data (e.g., blood glucose level) input icon. The blood data input icon is associated with a seventh, different time of the timeline 202 and is configured to receive user input as to one or more data points relating to the user's blood, such as current blood glucose level. As one example, the blood data input icon can present the user, at the diet challenge sequence 200, with a blood data (e.g., blood glucose) measurement request. As one specific example, the blood data input icon can present the user with a prompt instructing the user to measure one or more data points relating to the user's blood, and the user can then input, to the user device, the one or more data points relating to the user's blood (e.g., measured via a blood glucose meter and input via wired or wireless communication between the blood glucose meter and the user device or input via user input). In response to receiving user input as to the one or more data points relating to the user's blood as a result of the prompt, the blood data input icon can then display the user's measured blood parameter(s) (e.g., the measured blood glucose level). The user's input for blood data can then be transmitted via the network to the remote server and stored at the remote server for use in diet performance tracking analysis. Additionally, in some examples, the user's input for blood data can be displayed at the seventh diet challenge sequence icon 203G. Thus, blood data input from the user at the diet challenge sequence 200 can be transmitted via the network to the remote server and, in some examples, the seventh diet challenge sequence icon 203G can then display the input blood data (e.g., blood glucose measurement) at the seventh diet challenge sequence icon 203G on the timeline 202 (e.g., at a seventh, different time on the timeline 202).

The eighth diet challenge sequence icon 203H can be a food consumption icon. The food consumption icon is associated with an eighth, different time of the timeline 202 and is configured to receive user input as to the user's food consumption, for instance, at that eighth time on the timeline 202. The eighth diet challenge sequence icon 203H can thus be similar to, or the same as, that described previously for the third diet challenge sequence icon 203C and/or the fourth diet challenge sequence icon 203D, each of which can also be a food consumption icon.

FIG. 2D shows the fourth graphical user interface 220 of the diet challenge sequence 200 corresponding to the selected diet challenge—a ketosis diet challenge as the selected diet challenge in the illustrated embodiment. The fourth graphical user interface 220 can correspond to a fourth time period 201 in the diet challenge sequence 200. For example, here the fourth graphical user interface 220 corresponds to “Date X+3” which can be the fourth time period 201 in the ketosis diet challenge. For instance, the first time period in the ketosis diet challenge can be a first day of the ketosis diet challenge, the second time period in the ketosis diet challenge can be a second day of the ketosis diet challenge, the third time period in the ketosis diet challenge can be a third day of the ketosis diet challenge, and the fourth time period in the ketosis diet challenge can be a fourth day of the ketosis diet challenge. The timeline 202 can continue from the first graphical user interface 205 to the second graphical user interface 210 to the third graphical user interface 215 and to the fourth graphical user interface 220 since the sequence shown at the fourth graphical user interface 220 can be a continuance of the sequence shown at the first, second, and third graphical user interfaces 205, 210, 215.

In the illustrated embodiment, the diet challenge sequence icons 203 at the fourth graphical user interface 220 include a ninth diet challenge sequence icon 203I and a tenth diet challenge sequence icon 203J, and each of the ninth and tenth diet challenge sequence icons 203I, 203J is associated with the timeline 202. In particular, in this embodiment, each of the ninth and tenth diet challenge sequence icons 203I, 203J includes an indication as to a particular time on the timeline 202 where each of the diet challenge sequence icons 203I, 203J is located. Namely, the ninth diet challenge sequence icon 203I is located at a ninth time (e.g., “6:00 PM” of “Date X+3”) on the timeline 202, and the tenth diet challenge sequence icon 203J is located at a tenth, later time (e.g., “10:00 PM” of “Date X+3”).

As noted, each of the one or more diet challenge sequence icons 203 can correspond to a diet challenge sequence data point for the diet challenge sequence 200. For example, in the illustrated embodiment of the ketosis diet challenge, each of the ninth and tenth diet challenge sequence icons 203I, 203J can correspond to a ketosis diet challenge sequence data point for the ketosis diet challenge selected by the user.

For instance, the ninth diet challenge sequence icon 203I can be a food shopping icon. The food shopping icon is associated with a ninth, different time on the timeline 202. The food shopping icon is configured to provide the user with a list of food items to be purchased by the user. The list of food items to be purchased by the user and provided via the food shopping icon can be food items that are associated with the particular diet challenge that was previously selected by the user (e.g., food items that are associated with the selected ketosis diet challenge). As one example, the food shopping icon can include a list of specific food items curated by the instructor of the particular diet challenge that was previously selected by the user, and the instructor can choose the specific food items to be included on the list, provided via the food shopping icon, based on the particular diet challenge that was previously selected by the user. In particular, in this example, the instructor of the selected diet challenge can create a list of specific food items recommended to be consumed by the user and share this list of specific, recommended food items for consumption with the user via the diet challenge sequence 200 at the graphical user interface. User input at the food shopping icon can be transmitted via the network to the remote server and, in some examples, the remote server can be configured to transmit at least some of the user input to a food provider, such as a grocery store or food delivery service.

In another more specific such example, the food shopping icon can, in addition to the list of recommended food items to be purchased by the user, include a webpage link to a third party website from which the recommended food items can be purchased. For instance, the food shopping icon can provide the user with a webpage link to a grocery store, or restaurant delivery service, from which the user can submit a purchase order for the food items included in the list of specific food items recommended, by the instructor of the diet challenge, to be consumed by the user. In some examples, the food shopping icon can include a webpage link to an online shopping cart, which is prefilled, by the instructor of the diet challenge selected by the user, with specific food items associated with the diet challenge selected by the user. In this way, the food shopping icon can be configured to present the user with both recommended food items that are associated with the diet challenge selected by the user and a link to a grocery store or restaurant delivery service from which those recommended food items can be purchased. Indeed, in some cases the food shopping icon can be configured to present the user with a link to an online shopping cart at the grocery store or food delivery service's webpage with the online shopping cart prefilled (e.g., by the instructor of the diet challenge) with the recommended food items associated with the diet challenge selected by the user. As one illustrative example, if a user has previously selected the ketosis diet challenge, the food shopping icon can be configured to present the user with a link to an online shopping cart at the grocery store or food delivery service's webpage with the online shopping cart prefilled with food items associated with the previously selected ketosis diet challenge (e.g., ketchup with no sugar from the grocery store, or hamburger without the bun from a food delivery service).

Thus, the food shopping icon provided at the diet challenge sequence 200 can be useful in providing recommended food item purchases specific to the user. For example, the food shopping icon can be configured to present the user with recommended food item purchases based on the diet challenge previously selected by the user and, in some other examples, also based on one or more diet performance parameters. Accordingly, the food shopping icon can be configured to present the user with recommended food item purchases in a dynamic manner that is a function of the diet challenge previously selected by the user and/or one or more of that user's diet performance parameters. In this way, the food shopping icon can be configured to present a first user with a first list of recommended food item purchases based on the first user's previous selection of a first diet challenge and that user's first level of a first diet performance parameter (e.g., a first blood glucose level) and to present a second user with a second, different list of recommended food item purchases based on the second user's previous selection of a second, different diet challenge and/or that user's second, different level of the first diet performance parameter (e.g., a second, higher blood glucose level).

The tenth diet challenge sequence icon 203J can be a user exercise icon. The user exercise icon is associated with a tenth, different time on the timeline 202 and is configured to provide user exercise instruction and/or receive user exercise data as to the user's exercise, for instance, at that tenth time on the timeline 202. The tenth diet challenge sequence icon 203J can thus be similar to, or the same as, that described previously for the fifth and sixth diet challenge sequence icon 203E, 203F, which can each also be a user exercise icon.

The graphical user interface diet challenge sequence 200 can be useful in tracking user diet performance and providing diet coaching to the user in a manner that is specific to that user. For instance, the illustrated embodiment of the diet challenge sequence 200 can provide user exercise, food consumption, and food shopping instructions that are a function of the specific diet challenge previously selected by the user and/or one or more of that user's diet performance parameters previously input via the diet challenge sequence 200.

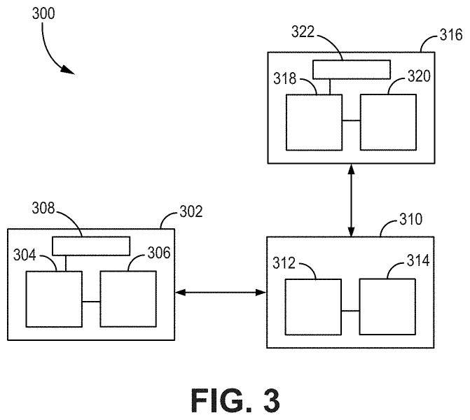

FIG. 3 illustrates an exemplary embodiment of a network 300 for implementing various embodiments disclosed herein. The network 300 can include a first user device 302, a remote server 310, and a diet challenge instructor device 316. In addition, in some embodiments, the network 300 can also include other, different user devices which can be similar to, or the same as, the first user device 302. The first user device 302 and remote server 310 can be in two-way data communication, and the diet challenge instructor device 316 and remote server 310 can be in two-way data communication. Thus, in some embodiments, the remote server 310 can facilitate data communication between the first user device 302 and the diet challenge instructor device 316.

The first user device 302 can include at least one programmable processor 304, a non-transitory computer-readable storage article 306, and a user interface 308. The non-transitory computer-readable storage article 306 can include computer-executable instructions stored thereon (e.g., the application described elsewhere herein) to cause the at least one programmable processor 304 to perform the functions described herein for various embodiments and provide the various graphical user interfaces described herein, for instance, via the user interface 308.

The remote server 310 can include at least one programmable processor 312 and a non-transitory computer-readable storage article 314. The non-transitory computer-readable storage article 314 can include computer-executable instructions stored thereon to cause the at least one programmable processor 312 to perform the functions described herein for various embodiments, including receiving data from the first user device 302 and/or diet challenge instructor device 316, analyzing data, and transmitting data to the first user device 302 and/or diet challenge instructor device 316.

The diet challenge instructor device 316 can include at least one programmable processor 318, a non-transitory computer-readable storage article 320, and a user interface 322. The non-transitory computer-readable storage article 320 can include computer-executable instructions stored thereon (e.g., the application described elsewhere herein) to cause the at least one programmable processor 318 to perform the functions described herein for various embodiments and provide the various graphical user interfaces described herein, for instance, via the user interface 322.

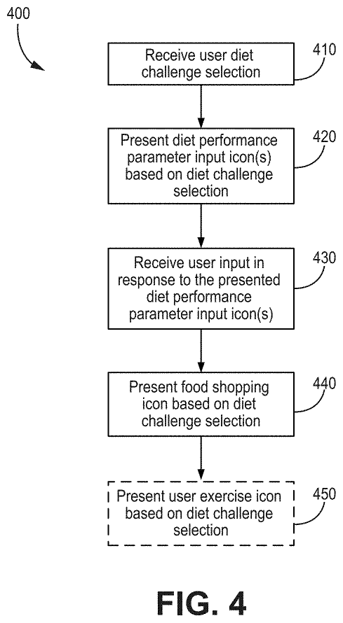

FIG. 4 is a flow diagram of an exemplary embodiment of a computer-implemented method 400 for providing a graphical user interface diet challenge sequence. As one example, the method 400 can be utilized to provide the graphical user interface diet challenge sequence shown for the exemplary embodiment illustrated at FIGS. 2A-2D.

At step 410, the method 400 includes receiving a user diet challenge selection. The diet challenge selection received at step 410 can be a first diet challenge selection from amongst a plurality of different diet challenges. For example, the diet challenge selection received at step 410 could be a ketosis diet challenge selected from amongst a plurality of other, different diet challenges.

At step 420, the method 400 includes presenting a diet performance parameter input icon based on the diet challenge selection received at step 410. As one example, the method can include creating a sub-group of one or more (e.g., two or more) diet performance parameter inputs from a main group of diet performance parameter inputs based on the diet challenge selection received at step 410, where the sub-group of one or more diet performance parameter inputs is different than the main group of diet performance parameter inputs (e.g., where the sub-group of one or more diet performance parameter inputs includes at least one less diet performance parameter than the main group of diet performance parameter inputs). And, at step 420, after the sub-group of the diet performance parameter input(s) is created based on the diet challenge selection received at step 410, this sub-group of the diet performance parameter input(s) can be presented. As a specific example, this can include creating a sub-group of a first diet performance parameter input (e.g., a ketone level input icon), a second diet performance parameter input (e.g., a mood input icon), and a third diet performance parameter input (e.g., a blood data input icon) from a main group of diet performance parameter inputs that includes the first, second, and third diet performance parameter inputs in addition to at least a fourth diet performance parameter input, where the first, second, third, and fourth diet performance parameter inputs are different from one another and the first, second, and third diet performance parameter inputs are selected for the sub-group based on the diet challenge selection received at step 410 (e.g., the fourth diet performance parameter is not associated with the diet challenge selection received at step 410). After the sub-group of the first, second, and third diet performance parameter inputs is created, this sub-group of the first, second, and third diet performance parameter inputs can be presented at the graphical user interface diet challenge sequence.

At step 430, the method 400 includes receiving user input in response to the diet performance parameter input icon(s) presented at step 420. For example, where the first diet performance parameter input icon presented is a ketone level input icon, user input received at step 430 can include user ketone level input. Likewise, where the second diet performance parameter input icon presented is a mood input icon, user input received at step 430 can include user mood input. And, where the third diet performance parameter input is a blood data input icon, user input received at step 430 can include user blood data input (e.g., user blood glucose level input).

At step 440, the method 400 includes presenting a food shopping icon based at least on the diet challenge selection received at step 410. The food shopping icon presented at step 440 can include a list of food items to be purchased by the user and associated with the particular diet challenge selection received at step 410, for instance as described previously herein in reference to the diet challenge sequence 203I. As one example described previously herein, presented food shopping icon can, in addition to the list of recommended food items to be purchased by the user, include a webpage link to a grocery store or food delivery service website from which the recommended food items can be purchased. For instance, the food shopping icon can present a webpage link to a grocery store, or restaurant delivery service, from which the user can submit a purchase order for the food items included in the list of specific food items presented via the food shopping icon. In some examples, the presented food shopping icon can include a webpage link to an online shopping cart, which is prefilled, by the instructor of the diet challenge selected by the user, with specific food items associated with the diet challenge selected by the user. As one illustrative example, if a user has previously selected the ketosis diet challenge at step 410, the food shopping icon can be configured to present the user with a link to an online shopping cart at the grocery store or food delivery service's webpage with the online shopping cart prefilled with food items associated with the previously selected ketosis diet challenge.

It can be useful, in some examples, to present the food shopping icon based at least on the diet challenge selection received at step 410. There can be significant physiological differences from one user to another, so presenting the food shopping icon based on the diet challenge selection received from the user at step 410 can allow for more effective associations between what food items are recommended via the presented food shopping icon and the specific diet challenge selected by the user.

In a further embodiment, at step 440, in addition to presenting the food shopping icon based on the diet challenge selection received at step 410, the food shopping icon can be presented based also on the user input received in response to the presented diet performance parameter input icon(s) at step 430. As one example, the list of recommended food items to be purchased by the user, and presented via the food shopping icon, can be selected by creating a sub-group from a larger main group by selecting those food items from the main group that are associated with the diet challenge selection received (at step 410). Then, food items from that sub-group can be selected based on the user input received in response to the presented diet performance parameter input icons (at step 430). As another example, the list of recommended food items to be purchased by the user, and presented via the food shopping icon, can be selected by creating a sub-group from a larger main group by selecting those food items from the main group based on the user input received in response to the presented diet performance parameter input icons (at step 430). Then, food items from that sub-group can be selected based on which of those in that sub-group are associated with the diet challenge selection received (at step 410).

As noted, presenting the food shopping icon at step 440 can include one or more actions described previously herein with reference to the diet challenge sequence icon 203I.

In some embodiments, the method 400 can include an additional step 450. When included in the method 400, the step 450 can include presenting a user exercise icon based at least on the diet challenge selection received at step 410. Presenting the user exercise icon based at least on the diet challenge selection received at step 410 can include one or more actions described previously herein with reference to the user exercise icons 203E, 203F, 203J.

Certain embodiments within the scope of the present disclosure can include an automated order feature. In particular, the automated order feature can determine when a particular item utilized by the user in connection with the diet challenge sequence has dropped below a predetermined unit quantity and, as a result, cause an order for additional units of that particular item to be placed without the user needing to manually place such order.

For example, as described previously, various diet challenge sequence icons can prompt a user biometric data input (e.g., ketone level input, blood glucose level input, etc.). Certain such biometric data inputs can be provided using a disposable biometric data substrate, such as a strip, that is input into a meter or other biometric data measuring device. For instance, to provide a user's ketone level input or blood glucose level, a user may provide a small amount of blood onto a disposable strip and then insert the strip, containing the blood, into a ketone and/or blood glucose meter so that the meter can measure the particular biometric data level (e.g., ketone level, blood glucose level, etc.). To facilitate the automated order feature, the number of biometric data inputs provided by a user can be tracked and counted. When the number of biometric data inputs provided by the user participating in the diet challenge sequence reaches a predetermined amount, the automated order feature can cause an order for additional units of the corresponding biometric data disposable to be placed without the user needing to manually enter an order for such additional biometric data disposables. As one specific example, a user can provide a ketone level input using a disposable ketone input strip input at a ketone meter. Each instance of a ketone level input can be counted, and when the number of ketone level inputs reaches a predetermined number, the automated order feature can cause an order for additional units of the disposable ketone input strip to be placed, via the network, without the user needing to manually enter an order for such additional disposable ketone input strips.

The automated order feature can be incorporated into the diet challenge sequence 200 and/or the method 400. For instance, the automated order feature can be embodied in computer-executable instructions along with the computer-executable instructions for providing the diet challenge sequence. As such, the corresponding computer-executable instructions for the automated order feature can be stored in a non-transitory computer-readable storage article, and the corresponding computer-executable instructions for the automated order feature can be stored on the non-transitory computer-readable storage article to cause at least one programmable processor to count each instance of a particular biometric data input involving a disposable unit and, upon the number of instances of the particular biometric data input involving the disposable unit reaching a predetermined number, the programmable processor can be caused to place an order, via the network, for additional units of the disposable unit without the user needing to manually enter an order for such additional disposable units. In some such examples, the predetermined number can correspond to a number of disposable units provided in a package minus a predetermined integer value (e.g., to allow for the order to be placed and delivered before the user exhausts all disposable units). Also, in some such examples, the diet challenge sequence 200 can include an automated order feature icon to indicate to the user that a disposable unit order has been automatically placed.

Various non-limiting exemplary embodiments have been described. It will be appreciated that suitable alternatives are possible without departing from the scope of the examples described herein.

What is claimed is:

1. A non-transitory computer-readable storage article having computer-executable instructions stored thereon to cause at least one programmable processor to:
receive, from a user, a diet challenge selection;
based on the diet challenge selection received, present via a graphical user interface at least one diet performance parameter icon associated with a timeline;
in response to the presented at least one diet performance parameter icon, receive user diet performance parameter input; and
based on the diet challenge selection received, present via the graphical user interface a food shopping icon associated with the timeline, wherein the food shopping icon is configured to provide a list of recommended food items to be purchased.

2. The article of claim 1, wherein at least three diet performance parameter icons, associated with the timeline, are presented via the graphical user interface based on the diet challenge selection received.

3. The article of claim 2, wherein the at least three diet performance parameter icons comprise a ketone level input icon, a mood input icon, and a blood data input icon.

4. The article of claim 3, wherein the ketone level input icon, the mood input icon, and the blood data input icon are presented, associated with the timeline, based on the diet challenge selection received being a ketosis diet challenge.

5. The article of claim 3, wherein the at least three diet performance parameter icons further comprise a user exercise icon, the user exercise icon configured to present the user with a prompt instructing the user to perform a specified exercise.

6. The article of claim 1, wherein the list of recommended food items to be purchased includes food items that are associated with the diet challenge selection received.

7. The article of claim 6, wherein the food shopping icon is further configured to provide a webpage link to a grocery store or food delivery service from which the list of recommended food items can be purchased.

8. The article of claim 7, wherein the webpage link is configured to direct a user to an online shopping cart prefilled with the list of recommended food items.

9. The article of claim 6, wherein the list of recommended food items to be purchased is created by creating a food item sub-group from a larger food item main group, wherein the food item sub-group is created from the larger food item main group by selecting those food items, from the main group to be included in the food item sub-group, that are associated with the diet challenge selection received.

10. The article of claim 9, wherein the diet challenge selection received is a ketosis diet challenge, and wherein the food item sub-group is created from the larger food item main group by selecting those food items, from the main group to be included in the food item sub-group, that are associated with the ketosis diet challenge.

11. A computer-implemented method for diet performance tracking, the method comprising the steps of:
receiving, from a user, a diet challenge selection;
based on the diet challenge selection received, presenting via a graphical user interface at least one diet performance parameter icon associated with a timeline;
in response to the presented at least one diet performance parameter icon, receiving user diet performance parameter input; and
based on the diet challenge selection received, presenting via the graphical user interface a food shopping icon associated with the timeline, wherein the food shopping icon provides a list of recommended food items to be purchased.

12. The method of claim 11, wherein at least three diet performance parameter icons, associated with the timeline, are presented via the graphical user interface based on the diet challenge selection received.

13. The method of claim 12, wherein the at least three diet performance parameter icons comprise a ketone level input icon, a mood input icon, and a blood data input icon.

14. The method of claim 13, wherein the ketone level input icon, the mood input icon, and the blood data input icon are presented, associated with the timeline, based on the diet challenge selection received being a ketosis diet challenge.

15. The method of claim 13, wherein the at least three diet performance parameter icons further comprise a user exercise icon, the user exercise icon configured to present the user with a prompt, via the graphical user interface, instructing the user to perform a specified exercise.

16. The method of claim 11, wherein the list of recommended food items to be purchased includes food items that are associated with the diet challenge selection received.

17. The method of claim 16, wherein the food shopping icon further provides a webpage link to a grocery store or food delivery service from which the list of recommended food items can be purchased.

18. The method of claim 17, wherein the webpage link directs the user to an online shopping cart prefilled with the list of recommended food items.

19. The method of claim 16, further comprising creating the list of recommended food items to be purchased by creating a food item sub-group from a larger food item main group, wherein the food item sub-group is created from the larger food item main group by selecting those food items, from the main group to be included in the food item sub-group, that are associated with the diet challenge selection received.

20. The method of claim 19, wherein the diet challenge selection received is a ketosis diet challenge, and wherein the food item sub-group is created from the larger food item main group by selecting those food items, from the main group to be included in the food item sub-group, that are associated with the ketosis diet challenge.
